(19) United States
(12) Patent Application Publication
(10) Pub. No.: US 2015/0126679 A1
(43) Pub. Date: May 7, 2015

(54) POLYMERIZABLE BENZOXAZINE COMPOUNDS WITH INTERFACIAL ACTIVE OR SURFACE ACTIVE PROPERTIES

(71) Applicant: HENKEL AG & CO. KGAA, Duesseldorf (DE)

(72) Inventors: Andreas Taden, Duesseldorf (DE); Stefan Kreiling, Duesseldorf (DE); Rainer Schoenfeld, Duesseldorf (DE)

(21) Appl. No.: 14/590,567

(22) Filed: Jan. 6, 2015

Related U.S. Application Data
(62) Division of application No. 13/241,416, filed on Sep. 23, 2011.
Application No. 13/241,416 is a continuation of application No. PCT/EP2010/056180, filed on May 6, 2010.

(30) Foreign Application Priority Data
May 12, 2009 (DE) 10 2009 003 033.6

(51) Int. Cl. C08G 73/06; C08L 101/00; C07D 413/06

(52) U.S. Cl. CPC C08G 73/06; C07D 413/06; C08L 101/00; C08L 2201/02; C08L 2201/54
USPC 524/612; 544/90; 528/403

(57) ABSTRACT

The invention relates to polymerizable benzoxazine compounds with interfacial active or surface active properties, having at least one polyalkylene oxide structural element, and to a method for producing said compounds. The invention also relates to benzoxazine (co)polymers comprising at least one of said benzoxazine compounds in the polymerized form.

The present invention relates to polymerizable benzoxazine compounds with interfacial active or surface active properties which have at least one polyalkylene oxide structural element, as well as a process for manufacturing the cited compounds. The invention further relates to benzoxazine (co)polymers that comprise the cited benzoxazine compounds in polymerized form.

Polymerizable benzoxazine compounds as well as benzoxazine(co)polymers as their polymerization products are known from the prior art.

Benzoxazine (co)polymers generally exhibit a high glass transition temperature and are characterized by their good electrical properties and their positive flame retardant behavior. Due to the poor solubility in aqueous media, in general neither the known benzoxazine (co)polymers nor established polymerizable benzoxazine compounds can be applied in the form of aqueous solutions, emulsions or dispersions.

However, for many application fields it is of critical importance that polymerizable benzoxazine compounds and the corresponding benzoxazine (co)polymers be applied in the form of aqueous presentation forms, as the use for example of volatile organic substances (VOC) in the application of the cited materials should be minimized from ecological considerations.

Thus, for example the German patent application DE 102005046546 A1 teaches curable mixtures based on benzoxazines, whose environmental compatibility is increased because the cited substances can be diluted with water. In spite of this there still remains a need to improve or to increase the environmental compatibility and the potential application of polymerizable benzoxazine compounds and of the corresponding benzoxazine (co)polymers, by improving their solubility in aqueous media.

The aim of the present invention was therefore to provide polymerizable benzoxazine compounds and the corresponding benzoxazine (co)polymers, which are highly soluble and/or easily dispersible in aqueous solutions and which therefore can be applied without using noxious organic solvents.

A first subject matter of the present invention is a polymerizable benzoxazine compound of the general Formula (I),

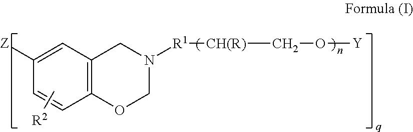

wherein q is a whole number from 1 to 4, n is a number from 2 to 20 000, R in each repeat unit is selected independently of each other from hydrogen or linear or branched, optionally substituted alkyl groups that comprise 1 to 8 carbon atoms, Z is selected from hydrogen (for q=1), alkyl (for q=1), alkylene (for q=2 to 4), carbonyl (for q=2), oxygen (for q=2), sulfur (for q=2), sulfoxide (for q=2), sulfone (for q=2) and a direct, covalent bond (for q=2), R¹ stands for a covalent bond or a divalent linking group that contains 1 to 100 carbon atoms,
R² is selected from hydrogen, halogen, alkyl and alkenyl, or R² is a divalent group that makes a corresponding naphthoxazine structure from the benzoxazine structure, Y is selected from linear or branched, optionally substituted alkyl groups that contain 1 to 15 carbon atoms, cycloaliphatic groups that optionally comprise one or more heteroatoms, alkyl groups that optionally comprise one or more heteroatoms and *—(C═O)R³, wherein R³ is selected from linear or branched, optionally substituted alkyl groups containing 1 to 15 carbon atoms and X—R⁴, wherein X is selected from S, O, and NH and R⁴ is selected from linear or branched, optionally substituted alkyl groups containing 1 to 15 carbon atoms.

A further subject matter of the present invention is a process for manufacturing the polymerizable benzoxazine compound according to the invention, comprising the step: treating at least one phenolic compound of the general Formula (IV),

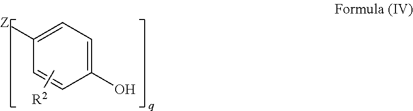

with at least one primary amine of the general Formula (V),

H₂N—R¹—(CH(R)—CH₂—O)ₙY Formula (V)

wherein q is a whole number from 1 to 4, n is a number from 2 to 20 000, Z is selected from alkylene (for q=2 to 4), carbonyl (for q=2), oxygen (for q=2), sulfur (for q=2), sulfoxide (for q=2), sulfone (for q=2) and a direct, covalent bond (for q=2) and R² is selected from hydrogen, halogen, alkyl and alkenyl, or R² is a divalent group that makes a corresponding naphthol structure from the phenol structure, R in each repeat unit is selected independently of each other from hydrogen or linear or branched, optionally substituted alkyl groups that comprise 1 to 8 carbon atoms, R¹ stands for a covalent bond or a divalent linking group that contains 1 to 100 carbon atoms, Y is selected from unbranched aliphatic alkyl groups that contain 1 to 15 carbon atoms, branched aliphatic alkyl groups that contain 1 to 15 carbon atoms, cycloaliphatic groups, cycloaliphatic groups that comprise one or more heteroatoms, aryl groups, aryl groups that comprise one or more heteroatoms and *-(C═O)R³, wherein R³ is selected from unbranched aliphatic alkyl groups that contain 1 to 15 carbon atoms, branched aliphatic alkyl groups that contain 1 to 15 carbon atoms and X—R⁴, wherein X is selected from S, O and NH and R⁴ is selected from unbranched aliphatic alkyl groups that contain 1 to 12 carbon atoms and branched aliphatic alkyl groups that contain 1 to 12 carbon atoms, with the proviso that the treatment is carried out in the presence of formaldehyde and/or a formaldehyde-releasing compound.

Likewise a subject matter of the present invention is a benzoxazine (co)polymer that comprises at least one inventive polymerizable benzoxazine compound in polymerized form.

The polymerizable benzoxazine compounds and the benzoxazine (co)polymers of the present invention generally exhibit a good solubility in aqueous media and therefore the cited substances can be employed in water-based formulations that are essentially free of organic solvents.

Moreover, the polymerizable benzoxazine compounds of the present invention possess surfactant properties, which is why these compounds can be employed as interfacial active or surface active substances in a large number of applications.

The benzoxazine (co)polymers according to the invention furthermore show a good capacity for interacting with a whole range of different surfaces, so that the cited polymers can be used for coating or modifying surfaces.

Consequently, further subject matters of the present invention are aqueous compositions or washing and cleaning agents as well as fabric treatment agents, which comprise at least one polymerizable benzoxazine compound and/or at least one benzoxazine (co)polymer, as well as the use of the cited benzoxazine compounds as a surfactant, in particular as a non-ionic surfactant.

Likewise subject matter of the present invention is the use of the benzoxazine (co)polymers according to the invention as a sizing agent for fibers, coating agent, for example as an antibacterial coating agent, as a corrosion protection agent and/or for improving the soil removal from and/or reducing the redeposition of soils on textiles or hard surfaces.

A final subject matter of the present invention is a process for treating and/or coating surfaces, wherein at least one surface is treated with at least one inventive polymerizable benzoxazine compound and/or with at least one inventive benzoxazine (co)polymer.

As already stated previously, the inventive polymerizable benzoxazine compound possess a chemical structure that is described by the general Formula (I),

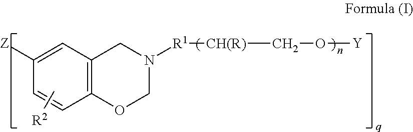

wherein q is a whole number from 1 to 4, n is a number from 2 to 20 000, R in each repeat unit is selected independently of each other from hydrogen or linear or branched, optionally substituted alkyl groups that comprise 1 to 8 carbon atoms, Z is selected from hydrogen (for q=1), alkyl (for q=1), alkylene (for q=2 to 4), carbonyl (for q=2), oxygen (for q=2), sulfur (for q=2), sulfoxide (for q=2), sulfone (for q=2) and a direct, covalent bond (for q=2), R¹ stands for a covalent bond or a divalent linking group that contains 1 to 100 carbon atoms, R² is selected from hydrogen, halogen, alkyl and alkenyl, or R² is a divalent group that makes a corresponding naphthoxazine structure from the benzoxazine structure, Y is selected from linear or branched, optionally substituted alkyl groups that contain 1 to 15 carbon atoms, cycloaliphatic groups that optionally comprise one or more heteroatoms, alkyl groups that optionally comprise one or more heteroatoms and *-(C═O)R³, wherein R³ is selected from linear or branched, optionally substituted alkyl groups containing 1 to 15 carbon atoms and X—R⁴, wherein X is selected from S, O, and NH and R⁴ is selected from linear or branched, optionally substituted alkyl groups containing 1 to 15 carbon atoms.

The divalent organic linking group R¹ in Formula (I) preferably contains 2 to 50, particularly preferably 2 to 25 and especially 2 to 20 carbon atoms. In addition, the divalent organic linking groups R¹ can each be selected from linear or branched, optionally substituted alkylene groups that contain 1 to 15 carbon atoms, wherein the alkylene groups are optionally interrupted by at least one heteroatom, selected from oxygen, sulfur or nitrogen. In the context of the present invention, the term “interrupted” is understood to mean that in a divalent alkylene group, at least one non-terminal carbon atom of said group is replaced by a heteroatom, wherein the heteroatom is preferably selected from *—S—* (sulfur), *—O—* (oxygen), and *—NR^{a}—* (nitrogen), wherein R^{a} stands in particular for hydrogen or for a linear or branched, optionally substituted alkyl group containing 1 to 15 carbon atoms.

The divalent organic compound group R¹ is preferably selected from alkylene groups that comprise 2 to 8 carbon atoms. In a preferred embodiment, R¹ is selected from linear alkylene groups that comprise 2 to 6, especially 2 or 3 carbon atoms, such as for example ethylene, propylene, butylene, pentylene and hexylene groups.

In one embodiment of the present invention, R¹ in Formula (I) stands for a covalent bond.

Moreover, the divalent organic compound group R¹ can comprise an arylene group and/or at least one biphenylene group that preferably each comprises 6 to 12 carbon atoms. The arylene groups and biphenylene groups can be substituted or unsubstituted, wherein suitable substituents are selected for example from alkyl, alkenyl, halogen, amine, thiol, carboxyl and hydroxyl groups. In addition, at least one carbon atom of the aromatic ring system of the cited groups can be replaced by a heteroatom, wherein the heteroatom is preferably selected from oxygen, nitrogen and sulfur.

In another embodiment of the invention, R in Formula (I) in each repeat unit is selected independently of each other from hydrogen or methyl.

In a preferred embodiment of the present invention, the polymerizable benzoxazine compounds of the general Formula (I) are selected from compounds of the general Formula (II),

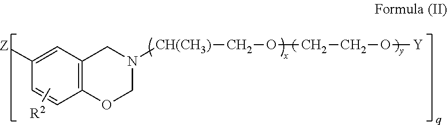

wherein x is a number between 0 and 1000 and y is a number between 0 and 1000, with the proviso that x+y≧2 and Z, R², Y and q are each defined as previously. Preferably, x+y≧3, particularly preferably ≧4 and quite particularly preferably ≧5. Depending on the application profile and the associated required solubility of the polymerizable benzoxazine compound of the general Formula (I) and (II) it is advisable to adjust the number of alkylene oxide units of the alkylene oxide chain in order to control the degree of hydrophilicity of the relevant compounds and their solubility behavior in various solvents. Generally, an increase in the number of the alkylene oxide units in the inventive benzoxazine compounds also leads to an increased solubility in water.

In specific embodiments of the invention, n or x+y therefore assumes as a lower limit a value of at least 3, 4, 6, 10, 12, 14, 16, 18, 20, 25, 30, 35, 40, 50, 60, 80, 100, 150 or 200. In the inventive benzoxazine compounds of the general Formula (I) or (II), an advantageous upper limit for n and/or x+y is preferably at a value of maximum 10 000, 2000, 1800, 1600, 1400, 1200, 1000, 800, 600 or 400.

The degree of hydrophilicity of the relevant compounds and their solubility behavior in various solvents can be controlled by the Y group that terminates the alkylene oxide chain in the polymerizable benzoxazine compounds of the general Formula (I) and (II). In one embodiment of the invention, Y in Formula (I) and/or Formula (II) stands for an alkyl group that comprises 1 to 8 carbon atoms, especially 1 to 6 carbon atoms, wherein Y preferably stands for a methyl group.

The inventive polymerizable benzoxazine compounds of the general Formula (I) and (II) exhibit good solubility in water-miscible alcohols, such as for example ethanol or propanol, in water itself or in any mixtures of the cited solvents.

In a specific embodiment of the invention, the solubility (at 20° C. and pH=7) of the inventive polymerizable benzoxazine compounds in water is at least 10 g/1000 g water.

In the present invention, the term “solubility” is understood to mean the maximum amount of a substance that the solvent (water) can take up at a defined temperature and a defined pH, i.e. the quantity of the dissolved substance in a saturated solution at the relevant temperature. If a solution comprises more dissolved substance than it should comprise in thermodynamic equilibrium at a defined temperature (e.g. when evaporating solvent), then the solution is called supersaturated. Seeding with seed crystals can cause for example the excess to precipitate out of the now simply saturated solution.

Supersaturated solutions should not be used when determining the solubility of the inventive polymerizable benzoxazine compounds. Methods to avoid the preparation of supersaturated solutions are known to the person skilled in the art. Likewise, suitable methods for determining the solubility of any substance are commonly used by the person skilled in the art.

In order to be suitable for the described application requirements, the inventive polymerizable benzoxazine compounds particularly advantageously have a solubility at 20° C. and at a pH of 7 of at least 10 g/1000 g water, preferably at least 50 g/1000 g water and particularly preferably 100 g/1000 g water.

As stated previously, a further subject matter of the present invention is a process for manufacturing the polymerizable benzoxazine compound according to the invention which process essentially comprises the following process step: treating at least one phenolic compound of the general Formula (IV),

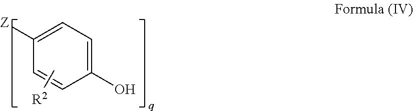

with at least one primary amine of the general Formula (V),

H₂N—R¹—(—CH(R)—CH₂—O—)ₙY Formula (V)

wherein q is a whole number from 1 to 4, n is a number from 2 to 20 000,
Z is selected from alkylene (for q=2 to 4), carbonyl (for q=2), oxygen (for q=2), sulfur (for q=2), sulfoxide (for q=2), sulfone (for q=2) and a direct, covalent bond (for q=2) and R² is selected from hydrogen, halogen, alkyl and alkenyl, or R² is a divalent group that makes a corresponding naphthol structure from the phenol structure,
R in each repeat unit is selected independently of each other from hydrogen or linear or branched, optionally substituted alkyl groups that comprise 1 to 8 carbon atoms, R¹ stands for a covalent bond or a divalent linking group that contains 1 to 100 carbon atoms, Y is selected from unbranched aliphatic alkyl groups that contain 1 to 15 carbon atoms, branched aliphatic alkyl groups that contain 1 to 15 carbon atoms, cycloaliphatic groups, cycloaliphatic groups that comprise one or more heteroatoms, aryl groups, aryl groups that comprise one or more heteroatoms and *-(C═O)R³, wherein R³ is selected from unbranched aliphatic alkyl groups that contain 1 to 15 carbon atoms, branched aliphatic alkyl groups that contain 1 to 15 carbon atoms and X—R⁴, wherein X is selected from S, O and NH and R⁴ is selected from unbranched aliphatic alkyl groups that contain 1 to 12 carbon atoms and branched aliphatic alkyl groups that contain 1 to 12 carbon atoms, with the proviso that the treatment is carried out in the presence of formaldehyde and/or a formaldehyde-releasing compound. Exemplary suitable phenolic compounds can be selected from mono- or biphenolic compounds, such as for example phenol, Bisphenol A, Bisphenol F, Bisphenol S or thiodiphenol. Formaldehyde itself, in addition to paraformaldehyde, trioxane or polyoxymethylene or any of their mixtures can also be used as the formaldehyde and/or a formaldehyde-releasing compound.

In a preferred embodiment of the primary amine of the general Formula (V) the divalent organic linking group R¹ preferably contains 2 to 50, particularly preferably 2 to 25 and especially 2 to 20 carbon atoms. In addition, each divalent organic linking group R¹ can be selected from linear or branched, optionally substituted alkylene groups that contain 1 to 15 carbon atoms, wherein the alkylene groups are optionally interrupted by at least one heteroatom, selected from oxygen, sulfur or nitrogen. In the context of the present invention, the term “interrupted” is understood to mean that in a divalent alkylene group, at least one non-terminal carbon atom of said group is replaced by a heteroatom, wherein the heteroatom is preferably selected from *—S—* (sulfur), *—O—* (oxygen), and *—NR^{a}—* (nitrogen), wherein R^{a} stands in particular for hydrogen or for a linear or branched, optionally substituted alkyl group containing 1 to 15 carbon atoms.

The divalent organic linking group R¹ is preferably selected from alkylene groups that contain 2 to 8 carbon atoms. In a preferred embodiment, R¹ is selected from linear alkylene groups that comprise 2 to 6, especially 2 or 3 carbon atoms, such as for example ethylene, propylene, butylene, pentylene and hexylene groups.

In one embodiment of the present invention, R¹ in Formula (V) stands for a covalent bond.

Moreover, the divalent organic compound group R¹ can comprise an arylene group and/or at least one biphenylene group that preferably each comprises 6 to 12 carbon atoms. The arylene groups and biphenylene groups can be substituted or unsubstituted, wherein suitable substituents are selected for example from alkyl, alkenyl, halogen, amine, thiol, carboxyl and hydroxyl groups. In addition, at least one carbon atom of the aromatic ring system of the cited groups can be replaced by a heteroatom, wherein the heteroatom is preferably selected from oxygen, nitrogen and sulfur.

In another embodiment of the invention, R in Formula (V) in each repeat unit is selected independently of each other from hydrogen or methyl.

In a preferred embodiment of the present invention, the primary amines of the general Formula (V) are selected from compounds of the general Formula (VI),

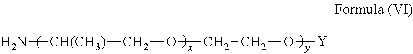

wherein x is a number between 0 and 1000 and y is a number between 0 and 1000, with the proviso that x+y≧2, wherein Y is defined as previously.
Preferably, x+y≧3, particularly preferably ≧4 and quite particularly preferably ≧5.

Depending on the application profile and the associated required solubility it is advisable to adjust the number of alkylene oxide units of the alkylene oxide chain. In specific embodiments of the invention, n and/or x+y in the primary amines therefore assumes as a lower limit a value of at least 3, 4, 6, 10, 12, 14, 16, 18, 20, 25, 30, 35, 40, 50, 60, 80, 100, 150 or 200.

In the primary amines of the general Formula (V) or (VI), an advantageous upper limit for n and/or x+y is preferably at a value of maximum 10 000, 2000, 1800, 1600, 1400, 1200, 1000, 800, 600 or 400.

In one embodiment of the invention, Y in Formula (V) and/or Formula (VI) stands for an alkyl group that comprises 1 to 8 carbon atoms, especially 1 to 6 carbon atoms, wherein Y preferably stands for a methyl group.

Particularly preferred primary amines of the general Formula (V) or (VI) are commercially available and are marketed by Huntsman Corp. Texas under the trade names Jeffamine® M-600, Jeffamine® M-1000, Jeffamine® M-2005, Jeffamine® M-2070, Jeffamine® M-2095 and Jeffamine® M-3000.

Another subject matter of the present invention is a benzoxazine (co)polymer that comprises at least one inventive polymerizable benzoxazine compound in polymerized form.

In the context of the present invention, a benzoxazine copolymer is understood to mean both a benzoxazine homopolymer as well as a benzoxazine copolymer. Benzoxazine homopolymers comprise only one inventive polymerizable benzoxazine compound in polymerized form, whereas benzoxazine copolymers contain, in addition to at least one inventive polymerizable benzoxazine compound, additional inventive polymerizable benzoxazine compounds and/or other polymerizable benzoxazine compounds.

The polymerization of the least one inventive polymerizable benzoxazine compound to a benzoxazine (co)polymer can be effected at increased temperatures following a self-initiation mechanism (thermal polymerization) or by adding cationic initiators. Suitable exemplary cationic initiators are Lewis acids or other cationic initiators, such as for example metal halides, organometallic reagents, such as metalloporphyrins, methyl tosylates, methyl triflates or trifluorosulfonic acids. Basic reagents can also be used in order to initiate the polymerization of the at least one inventive polymerizable benzoxazine compound. Suitable exemplary basic reagents can be selected from imidazole or imidazole derivatives.

The thermal polymerization of the at least one polymerizable benzoxazine compound according to the invention is preferably carried out at temperatures of 150° C. to 300° C., especially at temperatures of 160 to 220° C. The polymerization temperature can also be lower when the abovementioned initiators and/or other reagents are used.

The polymerization process is essentially based on the thermally induced ring opening of the oxazine ring of a benzoxazine system.

In a preferred embodiment of the present invention, the benzoxazine (co)polymer contains, in addition to an inventive polymerizable benzoxazine compound in polymerized form, at least one additional benzoxazine compound that is selected from compounds of the general Formula (III),

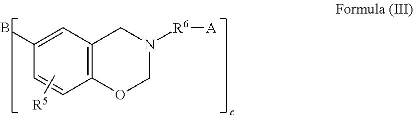

wherein c is a whole number from 1 to 4, B is selected from hydrogen (for c=1), alkyl (for c=1), alkylene (for c=2 to 4), carbonyl (for c=2), oxygen (for c=2), sulfur (for c=2), sulfoxide (for c=2), sulfone (for c=2) and a direct, covalent bond (for c=2), A is a hydroxyl group or a nitrogen-containing heterocycle, R⁵ is selected from hydrogen, halogen, alkyl and alkenyl, or R⁵ is a divalent group that makes a corresponding naphthoxazine structure from the benzoxazine structure and, R⁶ stands for a covalent bond or a divalent linking group that contains 1 to 100 carbon atoms.

The A group in Formula (III) stands for a hydroxyl group or a nitrogen-containing heterocycle. In the context of the present invention, the term “nitrogen-containing heterocycle” is understood to mean particularly those ring systems that comprise 3 to 8 ring atoms, preferably 5 to 6 ring atoms, wherein the ring system includes at least one nitrogen atom and at least two carbon atoms. Said nitrogen-containing heterocycle can have a saturated, unsaturated or aromatic structure and can also include additional heteroatoms, such as for example sulfur and/or oxygen atoms, in addition to the abovementioned atoms.

In accordance with Formula (III), the nitrogen-containing heterocycle is linked through the linking group R⁶ with the nitrogen atom of the oxazine ring of the benzoxazine structure. The divalent linking group R⁶ can be linked with each nitrogen or ring carbon atom of the nitrogen-containing heterocycle, in which R formally replaces a hydrogen atom that is covalently bonded to a nitrogen or ring carbon atom.

Exemplary particularly preferred nitrogen-containing heterocycles are selected from 5-membered nitrogen heterocycles, such as for example imidazoles, imidazolidones, tetrazoles, oxazoles, pyrroles, pyrrolidines and pyrazoles or 6-membered nitrogen-containing heterocycles, such as for example piperidines, piperidones, piperazines, pyridines, diazines and morpholines.

Preferred benzoxazine compounds of the general Formula (III) are in particular selected from compounds of the general Formula (VII) and/or from compounds of the general Formula (VIII),

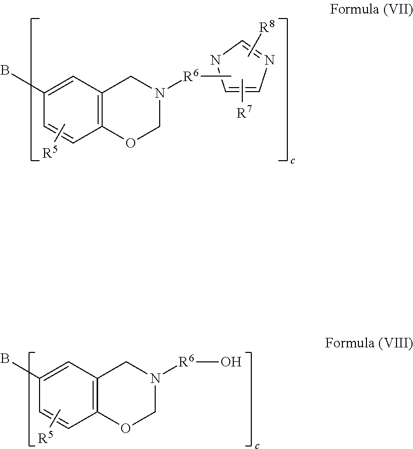

wherein R⁷ and R⁸ each independently of one another are selected from hydrogen, halogen, linear or branched, optionally substituted alkyl groups, alkenyl groups and aryl groups, wherein c, B, R⁵ and R⁶ are each as defined above.

In one embodiment of the invention, R⁷ and R⁸ in Formula (VII) are selected independently of one another from hydrogen, methyl, ethyl, n-propyl, iso-propyl, n-butyl, sec-butyl and iso-butyl, wherein R⁷ and R⁸ stand in particular for hydrogen or methyl.

Particularly preferred benzoxazine compounds of the general Formula (VII) are selected from the following benzoxazine compounds:

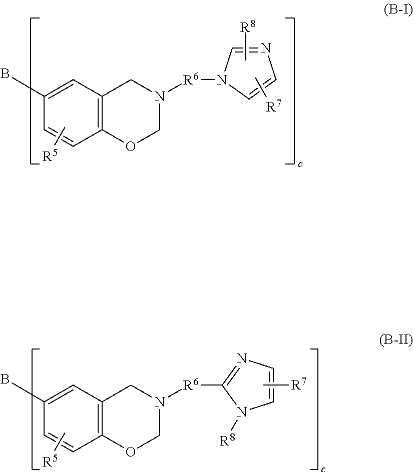

wherein c, B, R⁵, R⁶, R⁷ and R⁸ are defined as above.

Specific benzoxazine compounds of the general Formula (VII) can be selected for example from the following compounds:

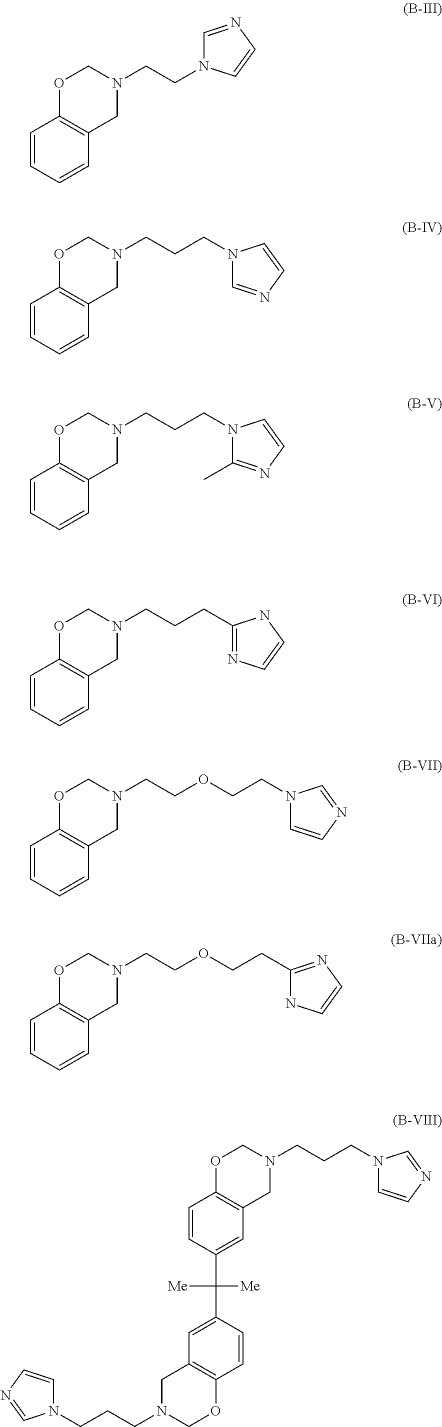

The illustrated benzoxazine compounds that carry an imidazole ring as the nitrogen-containing heterocycle can be obtained for example by treating a phenolic compound with an aldehyde, such as for example formaldehyde and an aminoalkyl-imidazole compound.

Exemplary suitable phenolic compounds can be selected from mono- or bisphenolic compounds, such as for example phenol, Bisphenol A, Bisphenol F, Bisphenol S or thiodiphenol.

Besides formaldehyde, paraformaldehyde, trioxane or polyoxymethylene or any of their mixtures can also be used as the aldehyde.

Preferred aminoalkyl-imidazole compounds have in particular a primary amino group and can be selected for example from compounds of the general Formula (A-I),

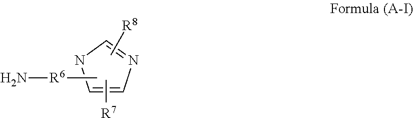

wherein R⁶, R⁷ and R⁸ are as described above.

In particular, 1-aminoalkyl-imidazole compounds of the general Formula (A-II),

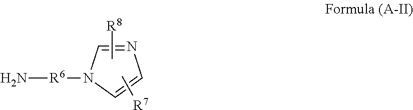

or 2-aminoalkyl-imidazole compounds of the general Formula (A-III)

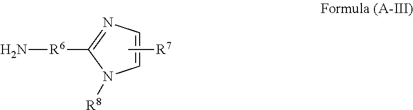

are suitable for manufacturing the corresponding benzoxazine compounds, wherein R⁶, R⁷ and R⁸ are as defined above.

In the context of the present invention, suitable 1-aminoalkyl-imidazole compounds of the general Formula (A-II) are known from the prior art and commercially available. Examples are for example 1-(3-aminopropyl)imidazole, available under the trade name Lupragen® API from BASF SE, 3-imidazol-1-yl-2-methyl-propylamine (Chem Pacific), 2-methyl-1H-imidazole-1-propanamine (3B Scientific Corporation), 3-imidazol-1-yl-2-hydroxy-propylamine (Ambinter, Paris Collection), 1-(4-aminobutyl)imidazole (Ambinter, Paris Collection), 2-ethyl-1H-imidazole-1-propanamine (ChemBridge Corp.).

Besides the use of commercially available 1-aminoalkyl-imidazole compounds of the general Formula (A-II), they can also be manufactured using well established synthetic organic methods, such as for example by a process that is described in Houben-Weyl, Methoden der organischen Chemie Vol. E 16d, Georg-Thieme-Verlag Stuttgart, 1992, pages 755 ff.

2-Aminoalkyl-imidazole compounds of the general Formula (A-III) are likewise known from the prior art. They can be manufactured using well established synthetic organic processes. A viable synthesis is described for example in Tetrahedron 2005, vol. 61, on pages 11148 to 11155.

Specific benzoxazine compounds of the general Formula (VIII) can be selected for example from the following compounds:

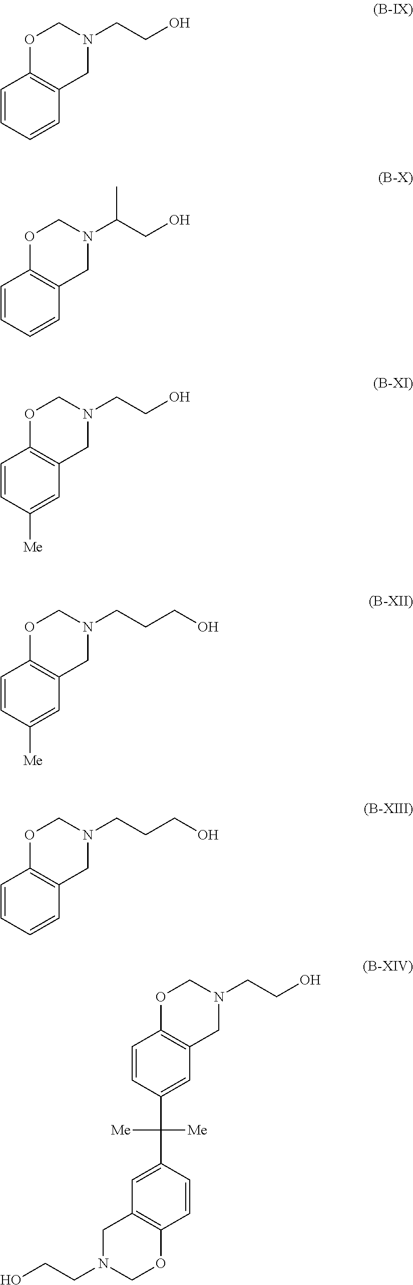

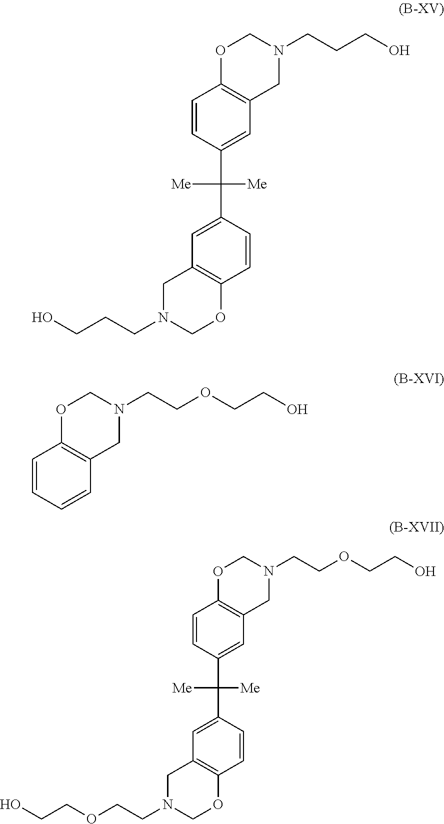

The illustrated benzoxazine compounds that carry a free hydroxyl group can be obtained from any well-established synthetic method, such as for example by a process that is described in the Japanese patent application JP 2002-302486 on page 11 in lines 66 to 100. The cited method is based on treating a phenolic compound with an aldehyde, such as for example formaldehyde and an amino alcohol. In this regard the reaction time can vary from some minutes up to some hours and depends strongly on the relative reactivity of the individual reactants.

Another method for manufacturing the illustrated benzoxazine compounds that carry a free hydroxyl group is described by Kiskan and Yagci in Polymer 46 (2005), pp. 11690 to 11697 and by Kiskan, Yagci and Ishida in the Journal of Polymer Science: Part A: Polymer Chemistry (2008), vol. 46, pp. 414-420.

Exemplary suitable phenolic compounds can be selected from mono- or bisphenolic compounds, such as for example phenol, Bisphenol A, Bisphenol F, Bisphenol S or thiodiphenol.

Besides formaldehyde, paraformaldehyde, trioxane or polyoxymethylene or any of their mixtures can also be used as the aldehyde.

Suitable amino alcohols, such as for example 2-aminoethanol, 3-amino-1-propanol, amino-2-propanol, 4-amino-1-butanol, 2-amino-1-butanol, 4-amino-2-butanol, 5-amino-1-pentanol, 6-amino-1-hexanol, 7-amino-1-heptanol, 3-amino-1,2-propane diol, 2-(2-aminoethoxy)ethanol and 2-amino-1,3-propane diol are commercially available and can be obtained for example from Sigma-Aldrich or Tokyo Chemical Industry.

The polymerizable benzoxazine compounds of the general Formula (III) can, in addition to the inventive polymerizable benzoxazine compounds of the general Formula (I), be used for manufacturing the benzoxazine (co)polymer of the present invention, wherein important material properties can be influenced by the relative mixing ratio of the individual polymerizable benzoxazine compounds.

Consequently, in one embodiment of the present invention, the benzoxazine (co)polymer includes in polymerized form
at least one inventive polymerizable benzoxazine compound of the general Formula (I), preferably at least one polymerizable benzoxazine compound of the general Formula (II) and
at least one polymerizable benzoxazine compound of the general Formula (VII) that carries at least one imidazole group.

The weight ratio of the at least one inventive polymerizable benzoxazine compound of the general Formula (I) to the at least one polymerizable benzoxazine compound of the general Formula (VII) in this case is preferably between 10:1 and 1:10, particularly preferably between 5:1 and 1:5 and in particular between 2:1 and 1:2, wherein for specific application purposes a weight ratio of 1:1 is advantageous.

In another embodiment of the present invention, the benzoxazine (co)polymer includes in polymerized form
at least one inventive polymerizable benzoxazine compound of the general Formula (I), preferably at least one polymerizable benzoxazine compound of the general Formula (II) and
at least one polymerizable benzoxazine compound of the general Formula (VIII) that carries at least one hydroxyl group.

The weight ratio of the at least one inventive polymerizable benzoxazine compound of the general Formula (I) to the at least one polymerizable benzoxazine compound of the general Formula (VIII) in this case is preferably between 10:1 and 1:10, particularly preferably between 5:1 and 1:5 and in particular between 2:1 and 1:2, wherein for specific application purposes a weight ratio of 1:1 is advantageous.

For particular applications it can make sense to use more than two different benzoxazine compounds for manufacturing the benzoxazine (co)polymer of the present invention. In a preferred embodiment the benzoxazine (co)polymer therefore includes in polymerized form
at least one polymerizable benzoxazine compound of the general Formula (I), preferably at least one polymerizable benzoxazine compound of the general Formula (II),
at least one polymerizable benzoxazine compound of the general Formula (VII) and
at least one polymerizable benzoxazine compound of the general Formula (VIII). The content of the individual benzoxazine compounds in the benzoxazine (co)polymer can vary in a broad range. Based on the total amount of the benzoxazine (co)polymer, the content of the benzoxazine compound (in polymerized form) of the general Formula (I) is preferably 5 to 90 wt. %, particularly preferably 10 to 80 wt. % and quite particularly preferably 25 to 50 wt. %; the content of the benzoxazine compound (in polymerized form) of the general Formula (VII) is preferably 5 to 90 wt. %, particularly preferably 10 to 80 wt. % and quite particularly preferably 25 to 50 wt. % and the content of the benzoxazine compound (in polymerized form) of the general Formula (VIII) is preferably 5 to 90 wt. %, particularly preferably 10 to 80 wt. % and quite particularly preferably 25 to 50 wt. %.

Moreover, it can be advantageous that the benzoxazine (co)polymer comprises, besides the already described benzoxazine compounds, additional polymerizable benzoxazine compounds in polymerized form, which differ from the abovementioned polymerizable benzoxazine compounds.

Suitable benzoxazine compounds are preferably represented by the Formula (B-XVIII),

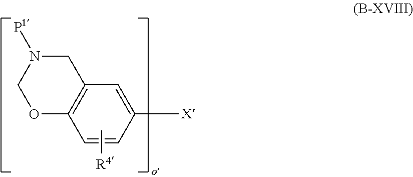

wherein o′ is a whole number between 1 and 4, X′ is selected from the group consisting of alkyl (for o′=1), alkylene (for o′=2 to 4), oxygen (for o′=2), thiol (for o′=1), sulfur (for o′=2), sulfoxide (for o′=2), sulfone (for o′=2) and a direct, covalent bond (for o′=2), R¹ is selected from the group consisting of hydrogen, alkyl, alkenyl and aryl and R⁴ is selected from the group consisting of hydrogen, halogen, alkyl and alkenyl, or R⁴ is a divalent group that makes a corresponding naphthoxazine structure from the benzoxazine structure.

Preferred benzoxazine compounds are in addition compounds of the general formula (B-IXX),

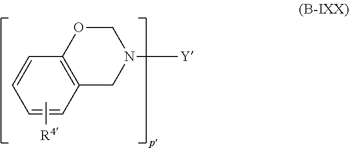

wherein p′=2 and Y is selected from the group consisting of biphenyl, diphenylmethane, diphenylisopropane, diphenyl sulfide, diphenyl sulfoxide, diphenyl sulfone, diphenyl ketone and R⁴ is selected from the group consisting of hydrogen, halogen, alkyl and alkenyl, or R⁴ is a divalent group that makes a corresponding naphthoxazine structure from the benzoxazine structure.

Likewise preferred benzoxazine compounds are in addition compounds of the general formula (B-XX) to (B-XXII),

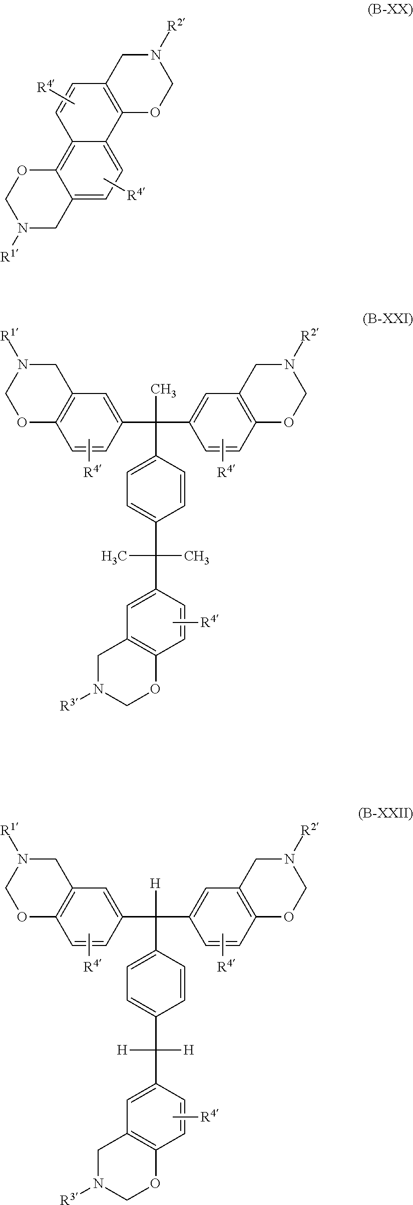

wherein R¹ and R⁴ are as defined above and R³ and R²′ are defined as R¹.

The illustrated benzoxazine compounds are commercially available and are marketed by inter alia Huntsman Advanced Materials; Georgia-Pacific Resins, Inc. and Shikoku Chemicals Corporation, Chiba, Japan.

Notwithstanding this, the benzoxazine compounds can also be obtained by treating a phenolic compound, for example Bisphenol A, Bisphenol F, Bisphenol S or thiophenol with an aldehyde, for example formaldehyde, in the presence of a primary amine.

Suitable manufacturing processes are described for example in U.S. Pat. No. 5,543,516, in particular disclosed in the examples 1 to 19 in columns 10 to 14, wherein the reaction time of the relevant reaction can take some minutes to some hours, depending on the concentration, reactivity and reaction temperature. Additional manufacturing possibilities can be taken from the U.S. Pat. Nos. 4,607,091, 5,021,484, 5,200,452 and 5,443,911.

The weight average molecular weight “Mw” of the benzoxazine (co)polymers is preferably between 500 and 100 000 g/mol, particularly preferably between 1000 and 100 000 g/mol and quite particularly preferably between 3000 and 50 000 g/mol. In this regard the weight average molecular weight can be measured by means of gel permeation chromatography (GPC) with a polystyrene standard.

The structure of the inventive benzoxazine (co)polymer is linear or branched depending on the choice of the benzoxazine compounds. Linear structures are preferred due to their high water-solubility and their good capacity for interaction with a large number of surfaces.

The water-solubility of the benzoxazine (co)polymer of the present invention can be further increased in a targeted manner by converting the cited polymers into their protonated forms by treatment with suitable acids. Protonated benzoxazine (co)polymers, whose solubility behavior is optimized for a predetermined application, can be obtained by varying the degree of protonation, for example by means of the concentration and the strength of the added acid.

A further subject matter of the present invention is an aqueous composition that comprises at least one inventive polymerizable benzoxazine compound and/or at least one benzoxazine (co)polymer of the present invention. The use of said benzoxazine compounds or benzoxazine (co)polymers in aqueous compositions is advantageous, as the described substances each display interfacial active or surface active properties and therefore can be used as an emulsifier or as a surfactant, in particular as a niosurfactant (non-ionic surfactant).

A further subject matter of the present invention is therefore also a washing and cleaning agent that comprises at least one inventive polymerizable benzoxazine compound and/or at least one benzoxazine (co)polymer of the present invention, as well as the use of said compounds as surfactants, in particular as niosurfactants.

The content of the at least one inventive polymerizable benzoxazine compound and/or the at least one benzoxazine (co)polymer of the present invention in the aqueous composition or in the fabric or surface treatment agent should be determined such that the surface treated with said agent is adequately covered. The quantity of the at least one polymerizable benzoxazine compound and/or the at least one benzoxazine (co)polymer of the present invention in the total amount of the finished agent is preferably 0.01 to 20 wt. %, particularly preferably 0.1 to 10 wt. % and especially 0.5 to 5 wt. %.

The fabric or surface treatment agent of the present invention particularly concerns agents that are liquid or in gel form.

The fabric or surface treatment agent of the present invention also comprises surfactants in addition to the inventive benzoxazine (co)polymers or the mixture of different benzoxazine (co)polymers, wherein said surfactants are particularly selected from anionic, cationic, ampholytic and non-ionic surfactants as well as from any of their mixtures.

Generally, anionic surfactants contain a water solubilizing anionic group, such as e.g. a carboxylate, sulfate, sulfonate or phosphate group and a lipophilic alkyl group containing about 8 to 30 carbon atoms. In addition, the molecule may comprise glycol or polyglycol ether groups, ester, ether and amide groups as well as hydroxyl groups. Exemplary suitable anionic surfactants are, each in the form of the sodium, potassium and ammonium as well as the mono, di and trialkanolammonium salts containing 2 to 4 carbon atoms in the alkanol group,
linear and branched fatty acids with 8 to 30 carbon atoms (soaps),
ether carboxylic acids of the formula R¹³—O—(CH₂—CH₂)ₓ—CH₂—COOH, in which R¹³ is a linear alkyl group with 8 to 30 carbon atoms and x=0 or 1 to 16,
acyl sarcosides with 8 to 24 carbon atoms in the acyl group,
acyl taurides with 8 to 24 carbon atoms in the acyl group,
acyl isethionates with 8 to 24 carbon atoms in the acyl group,
mono and dialkyl esters of sulfosuccinic acid containing 8 to 24 carbon atoms in the alkyl group and
mono-alkyl polyoxyethyl esters of sulfosuccinic acid with 8 to 24 carbon atoms in the alkyl group and 1 to 6 oxyethylene groups,-linear alpha-olefin sulfonates with 8 to 24 carbon atoms,
alpha-sulfo fatty acid methyl esters of fatty acids containing 8 to 30 carbon atoms,
alkyl sulfates and alkyl polyglycol ether sulfates of the Formula R¹⁴—O(CH₂—CH₂O)ₓ—OSO₃H, in which R¹⁴ is preferably a linear alkyl group containing 8 to 30 carbon atoms and x=0 or 1 to 12,
mixtures of surface active hydroxyl sulfonates,
sulfated hydroxyalkyl polyethylene glycol ethers and/or hydroxyalkylene propylene glycol ethers,
sulfonates of unsaturated fatty acids with 8 to 24 carbon atoms and 1 to 6 double bonds,
esters of tartaric acid and citric acid with alcohols, which represent the addition products of about 2-15 molecules of ethylene oxide and/or propylene oxide on fatty alcohols containing 8 to 22 carbon atoms,
alkyl and/or alkenyl ether phosphates of Formula (E1-I),

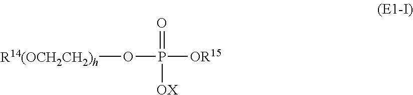

in which R¹⁴ preferably stands for an aliphatic hydrocarbon group containing 8 to 30 carbon atoms, R¹⁵ stands for hydrogen, a (CH₂CH₂O)ₙR¹⁶ group or X, h for numbers between 1 and 10 and X for hydrogen, an alkali- or alkaline earth metal or NR¹⁷R¹⁸R¹⁹R²⁰, with R¹⁷ to R¹⁹, independently of each other standing for a C₁ to C₄ hydrocarbon group,
sulfated fatty acid alkylene glycol esters of Formula (E1-II)

R²⁰CO(AlkO)ₙSO₃M (E1-II)

in which R²⁰CO— stands for a linear or branched, aliphatic, saturated and/or unsaturated acyl group with 6 to 22 carbon atoms, Alk for CH₂CH₂, CHCH₃CH₂ and/or CH₂CHCH₃, n for numbers from 0.5 to 5 and M for a cation,
monoglyceride sulfates and monoglyceride ether sulfates of Formula (E1-III)

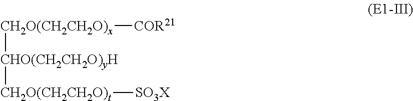

in which R²¹CO stands for a linear or branched acyl group containing 6 to 22 carbon atoms, the sum of x, y and i is 0 or stands for numbers between 1 and 30, preferably 2 to 10, and X stands for an alkali metal or alkaline earth metal. In the context of the invention, typical examples of suitable monoglyceride (ether) sulfates are the reaction products of lauric acid monoglyceride, cocoa fatty acid monoglyceride, palmitic acid monoglyceride, stearic acid monoglyceride, oleic acid monoglyceride and tallow fatty acid monoglyceride as well as their ethylene oxide adducts with sulfur trioxide or chlorosulfonic acid in the form of their sodium salts. Preferably, monoglyceride sulfates of Formula (E1-III) are employed, in which R²¹CO stands for a linear acyl group containing 8 to 18 carbon atoms,
amido ether carboxylic acids,
condensation products of C₈-C₃₀ fatty alcohols with protein hydrolyzates and/or amino acids and their derivatives, which are known to the person skilled in the art as albumin fatty acid condensates, such as for example the Lamepon® types, Gluadin® types, Hostapon® KCG or the Amisoft® types.

Preferred anionic surfactants are alkyl sulfates, alkyl polyglycol ether sulfates and ether carboxylic acids with 10 to 18 C atoms in the alkyl group and up to 12 glycol ether groups in the molecule, sulfosuccinic acid mono and dialkyl esters with 8 to 18 C atoms in the alkyl group and sulfosuccinic acid mono-alkyl polyoxyethyl esters with 8 to 18 C atoms in the alkyl group and 1 to 6 oxyethylene groups, monoglycerin disulfates, alkyl- and alkenyl ether phosphates as well as albumin fatty acid condensates.

According to the invention, cationic surfactants of the type quaternary ammonium compounds, the esterquats and the amido amines are preferred. Preferred quaternary ammonium compounds are ammonium halides, particularly chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known under the INCI names Quaternium-27 and Quaternium-83. The long alkyl chains of the abovementioned surfactants have preferably 10 to 18 carbon atoms.

Esterquats are known compounds, which both comprise at least one ester function and also a quaternary ammonium group as structural elements. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines. Such products are marketed, for example, under the trade names Stepantex®, Dehyquart® and Armocare®. The products Armocare® VGH-70, an N,N-bis(2-palmitoyloxyethyl)dimethylammonium chloride, as well as Dehyquart® F-75, Dehyquart® C-4046, Dehyquart® L80 and Dehyquart® AU 35 are examples of such esterquats.

The alkylamido amines are normally manufactured by the amidation of natural or synthetic fatty acids and fatty acid fractions with dialkylamino amines. According to the invention, a particularly suitable compound from this substance group is represented by stearamidopropyldimethylamine, commercially available under the designation Tegamid® S 18.

In addition to or instead of the cationic surfactants, the agents can comprise further surfactants or emulsifiers, wherein in principle both anionic as well as ampholytic and non-ionic surfactants and all types of known emulsifiers are suitable. The group of the ampholytic or also amphoteric surfactants includes zwitterionic surfactants and ampholytes. The surfactants can already have an emulsifying action.

Zwitterionic surfactants are designated as those surface-active compounds that carry at least one quaternary ammonium group and at least one —COO⁽⁻⁾ or —SO₃⁽⁻⁾ group in the molecule. Particularly suitable zwitterionic surfactants are the so-called betaines such as the N-alkyl-N,N-dimethylammonium glycinates, for example the cocoalkyl dimethylammonium glycinate, N-acylaminopropyl-N,N-dimethylammonium glycinates, for example the cocoacylaminopropyl dimethylammonium glycinate, and 2-alkyl-3-carboxymethyl-3-hydroxyethyl imidazolines with 8 to 18 carbon atoms in each of the alkyl or acyl groups, as well as cocoacylaminoethyl hydroxyethyl carboxymethyl glycinate. A preferred zwitterionic surfactant is the fatty acid amide derivative, known under the INCI name Cocamidopropyl Betaine.

Ampholytes are understood to include such surface-active compounds that apart from a C₈₋₂₄ alkyl or acyl group, comprise at least one free amino group and at least one —COOH or —SO₃H group in the molecule, and are able to form internal salts. Examples of suitable ampholytes are N-alkylglycines, N-alkyl propionic acids, N-alkylamino butyric acids, N-alkylimino dipropionic acids, N-hydroxyethyl-N-alkylamidopropylglycines, N-alkyltaurines, N-alkylsarcosines, 2-alkylamino propionic acids and alkylamino acetic acids, each with about 8 to 24 carbon atoms in the alkyl group. Particularly preferred ampholytes are N-cocoalkylamino propionate, cocoacylaminoethylamino propionate and C₁₂-C₁₈ acyl sarcosine.

Non-ionic surfactants comprise e.g. a polyol group, a polyalkylene glycol ether group or a combination of polyol ether groups and polyglycol ether groups as the hydrophilic group. Exemplary compounds of this type are
addition products of 2 to 50 moles ethylene oxide and/or 1 to 5 moles propylene oxide to linear and branched fatty alcohols containing 8 to 30 carbon atoms, to fatty acids containing 8 to 30 carbon atoms and to alkyl phenols containing 8 to 15 carbon atoms in the alkyl group,
methyl or C₂-C₆ alkyl group end blocked addition products of 2 to 50 moles ethylene oxide and/or 1 to 5 moles propylene oxide to linear and branched fatty alcohols with 8 to 30 carbon atoms, to fatty acids with 8 to 30 carbon atoms and to alkyl phenols with 8 to 15 carbon atoms in the alkyl group, such as, for example, the commercially available types Dehydrol® LS, Dehydrol® LT (Cognis),
C₁₂-C₃₀ fatty acid mono- and diesters of addition products of 1 to 30 moles ethylene oxide to glycerin,
addition products of 5 to 60 moles ethylene oxide on castor oil and hydrogenated castor oil,
polyol esters of fatty acids, such as, for example, the commercial product Hydagen® HSP (Cognis) or Sovermol types (Cognis),
alkoxylated triglycerides,
alkoxylated fatty acid alkyl esters of the formula (E4-I)

R²²CO—(OCH₂CHR²³)_{w}OR²⁴ (E4-I)

in which R²²CO stands for a linear or branched, saturated and/or unsaturated acyl group containing 6 to 22 carbon atoms, R²³ for hydrogen or methyl, R²⁴ for linear or branched alkyl groups containing 1 to 4 carbon atoms and w for numbers from 1 to 20,
amine oxides,
hydroxy mixed ethers,
sorbitol esters of fatty acids and addition products of ethylene oxide to sorbitol esters of fatty acids such as e.g. the polysorbates,
sugar esters of fatty acids and addition products of ethylene oxide to sugar esters of fatty acids,
addition products of ethylene oxide to fatty acid alkanolamides and fatty amines,
sugar surfactants of the type alkyl and alkenyl oligoglycosides according to Formula (E4-II),

R²⁵O[G]ₚ (E4-II)

in which R²⁵ stands for an alkyl or alkenyl group containing 4 to 22 carbon atoms, G for a sugar group containing 5 or 6 carbon atoms and p for numbers from 1 to 10. They can be obtained according to the appropriate methods of preparative organic chemistry. The alkyl and alkenyl oligoglycosides can derive from aldoses or ketoses containing 5 or 6 carbon atoms, preferably from glucose. The preferred alkyl and/or alkenyl oligoglycosides are accordingly alkyl and/or alkenyl oligoglucosides The index value p in the general Formula (E4-II) represents the degree of oligomerization (DP), i.e. the distribution of mono and oligoglycosides, and stands for a number between 1 and 10. Whereas in a single molecule, p must always be a whole number and here above all can assume the values p=1 to 6, the value p for a specific alkyl oligoglycoside is an analytically determined, calculated quantity that mostly represents a fractional number. Preferably, alkyl and/or alkenyl oligoglycosides are employed with an average degree of oligomerization p of 1.1 to 3.0. From the industrial point of view, such alkyl and/or alkenyl oligoglycosides are preferred with degrees of oligomerization less than 1.7 and in particular between 1.2 and 1.4. The alkyl or alkenyl group R²⁵ can be derived from primary alcohols containing 4 to 11, preferably 8 to 10 carbon atoms. Typical examples are butanols, caproyl alcohol, caprylic alcohol, capric alcohol and undecyl alcohol as well as their industrial mixtures, such as for example those obtained by the hydrogenation of industrial fatty acid methyl esters or in the course of the hydrogenation of aldehydes from the Roelen Oxo-synthesis. Alkyl oligoglucosides with chain lengths C₈-C₁₀ (DP=1 to 3) are preferred, which result as the low boiling fraction in the separative distillation of industrial C₈-C₁₈ coco fatty alcohol and which can be contaminated with a fraction of less than 6 wt. % of C₁₋₂ alcohol, as well as alkyl oligoglucosides based on industrial C₉₁₁₁ oxo alcohols (DP=1 to 3). The alkyl or alkenyl group R²⁵ can moreover be derived from primary alcohols containing 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol as well as their industrial mixtures that can be obtained as described above. Alkyl oligoglucosides based on hydrogenated C_{12/14} coco alcohol with a DP of 1 to 3 are preferred.
sugar surfactants of the type fatty acid N-alkylpolyhydroxyalkyl amides, a non-ionic surfactant of Formula (E4-III),

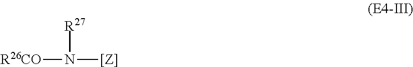

in which R²⁶CO stands for an aliphatic acyl group with 6 to 22 carbon atoms, R²⁷ for hydrogen, an alkyl or hydroxyalkyl group with 1 to 4 carbon atoms and [Z] for a linear or branched polyhydroxyalkyl group with 3 to 12 carbon atoms and 3 to 10 hydroxyl groups. The fatty acid N-alkylpolyhydroxyalkyl amides are known substances, which may normally be obtained by reductive amination of a reducing sugar with ammonia, an alkylamine or an alkanolamine and subsequent acylation with a fatty acid, a fatty acid alkyl ester or a fatty acid chloride. The fatty acid N-alkylpolyhydroxyalkyl amides are advantageously derived from reducing sugars having 5 or 6 carbon atoms, especially from the glucoses. Accordingly, the fatty acid N-alkyl glucamides illustrate the fatty acid N-alkylpolyhydroxyalkyl amides, as are shown by the Formula (E4-IV):

R²⁸CO—NR²⁹—CH₂—(CHOH)₄CH₂OH (E4-IV)

Preferably, glucamides of the Formula (E4-IV) are employed as the fatty acid N-alkylpolyhydroxyalkyl amides, in which R²⁹ stands for hydrogen or an alkyl group and R²⁸CO stands for the acyl group of caproic acid, caprylic acid, capric acid, lauric acid, myristic acid, palmitic acid, palmoleic acid, stearic acid, isostearic acid, oleic acid, elaidic acid, petroselic acid, linoleic acid, linolenic acid, arachic acid, gadoleic acid, behenic acid or erucic acid or their industrial mixtures. Fatty acid N-alkylglucamides of Formula (E4-IV) are particularly preferred, which are obtained by reductive amination of glucose with methylamine and subsequent acylation with lauric acid or C_{12/14} coco fatty acid or a corresponding derivative. In addition, the polyhydroxyalkyl amides can also derive from maltose and palatinose.

Alkylene oxide addition products to saturated, linear fatty alcohols and fatty acids, each with 2 to 30 moles ethylene oxide per mole fatty alcohol or fatty acid, have proved to be preferred non-ionic surfactants. Agents with excellent properties are also obtained when they comprise fatty acid esters of ethoxylated glycerine as the non-ionic surfactants.

These compounds are characterized by the following parameters. The alkyl group comprises 6 to 22 carbon atoms and may be both linear and also branched. Primary linear aliphatic groups and aliphatic groups that are methyl-branched in the 2-position, are preferred. Such alkyl groups are for example 1-octyl, 1-decyl, 1-lauryl, 1-myristyl, 1-cetyl and 1-stearyl. 1-Octyl, 1-decyl, 1-lauryl, 1-myristyl are particularly preferred. On using so-called “oxo alcohols” as the starting materials, compounds with an odd number of carbon atoms in the alkyl chain preponderate.

The sugar surfactants can also be comprised as the non-ionic surfactants. For compounds with alkyl groups that are used as surfactants, they may each be pure substances. However, it is normally preferred to start with natural vegetal or animal raw materials for the manufacture of these materials, with the result that mixtures of substances are obtained, which have different alkyl chain lengths that depend on each raw material. For surfactants, which are represented by the addition products of ethylene oxide and/or propylene oxide to fatty alcohols or derivatives of these addition products, both products with a “normal” homologue distribution as well as those with a narrow homologue distribution may be used. The term “normal” homologue distribution is understood to mean mixtures of homologues obtained from the reaction of fatty alcohols and alkylene oxide using alkali metals, alkali metal hydroxides or alkali metal alkoxides as catalysts. On the other hand, narrow homologue distributions are obtained if e.g. hydrotalcite, alkaline earth metal salts of ether carboxylic acids, alkaline earth metal oxides, hydroxides or alkoxides are used as the catalysts. The use of products with a narrow homologue distribution can be preferred.

The amount of surfactant in the inventive fabric or surface treatment agent depends strongly on the proposed application and is preferably in a range of 0.5 to 50 wt. %, particularly preferably in a range of 0.5 to 35 wt. % and quite particularly preferably in a range of 1 to 10 wt. %, each based on the total weight of the agent.

Furthermore, the fabric or surface treatment agent of the present invention can comprise at least one fragrance that preferably lends the agent a pleasant and/or fresh fragrant impression. The at least one fragrance is not subject to any limitations. Thus, individual odoriferous compounds, both synthetic or natural products of the ester, ether, aldehyde, ketone, alcohol, hydrocarbon, acid, carboxylic acid ester, aromatic hydrocarbon, aliphatic hydrocarbon type, saturated and/or unsaturated hydrocarbons and mixtures thereof can be used as the at least one fragrance.

As the fragrance aldehydes or fragrance ketones, all usual fragrant aldehydes and fragrant ketones can be employed which are typically used to procure a pleasant fragrant sensation. Suitable fragrant aldehydes and fragrant ketones are generally known to the person skilled in the art. The total quantity of the at least one fragrance in the inventive agent for fabric or surface treatment is preferably between 0.01 and 5 wt. %, particularly preferably between 0.1 and 3 wt. % and quite particularly preferably between 0.5 and 2 wt. % based on the total quantity of the agent.

Mixtures of various fragrances (from the various fragrances cited above), which together produce an attractive fragrant note, are preferably used. In this case the total quantity of the at least one fragrance is the quantity of all fragrances together in the mixture based on the total quantity of the composition.

In a preferred development of the present invention, the agent for fabric or surface treatment concerns a fabric treatment agent that for example can be employed for fabric pretreatment as well as for fabric after treatment and for fabric washing. The agent for fabric treatment can be employed both in the private segment as well as in the textile industry, wherein the benzoxazine (co)polymers according to the invention can be used both for permanent as well as for temporary fabric treatment.

In a most preferred embodiment of the invention, said fabric treatment agent is a washing agent, fabric softener, softening washing agent or washing auxiliary, wherein said agents can comprise, in addition to the already mentioned ingredients, additional ingredients, such as for example builders, bleaching agents, bleach activators, enzymes, electrolytes, non-aqueous solvents, pH adjustors, perfume carriers, fluorescent agents, colorants, hydrotropes, foam inhibitors, silicone oils, anti-redeposition agents, optical brighteners, anti-graying inhibitors, antimicrobials, germicides, fungicides, antioxidants, preservatives, corrosion inhibitors, antistats, bittering agents, ironing aids, water-repellents and impregnation agents, swelling and non-skid agents, neutral filler salts and UV-absorbers.

A subject matter of the present invention is likewise a process for treating and/or coating surfaces, wherein at least one surface is treated with at least one benzoxazine (co)polymer of the present invention.

The benzoxazine (co)polymer of the present invention is preferably deposited onto the relevant surface in the form of an aqueous solution, dispersion or emulsion. In particular, aqueous solutions, dispersions or emulsions are preferred which have a water content of at least 5 wt. %, preferably at least 50 wt. % and particularly preferably at least 90 wt. %, based on the total amount of the agent. Likewise, alcohol-based solutions, dispersions or emulsions are preferred which have an alcohol content of at least 5 wt. %, preferably at least 50 wt. % and particularly preferably at least 90 wt. %, based on the total amount of the agent. In particular, preferred alcohols are selected from ethanol, isopropanol or from any of their mixtures. Furthermore, the cited solutions, dispersions or emulsions can also comprise any mixtures of water and water-miscible alcohols, such as for example water/ethanol and water/propanol mixtures.

Different surfaces can be furnished with different properties by the treatment with the inventive benzoxazine (co)polymer or with an agent that contains the inventive benzoxazine (co)polymer.

In this regard, preferred surfaces are selected from carbon fibers, hard surfaces and fabric surfaces.

Carbon fibers are used inter alia for manufacturing fiber-reinforced composites. Fiber-reinforced composites generally consist as mixed materials of at least two components. In addition to a resin component, the fiber-reinforced composites contain a carbon fiber component that can consist for example of unidirectional as well as of web or short fibers. The carbon fiber component combined with the added resin component lends the material a high strength, which is why fiber-reinforced composites are employed as composites in application fields with high demands for structural material properties, such as for example in aircraft or automobile construction. In order to form a high quality and stable fiber-reinforced composite on an industrial scale, many carbon bundles, composed of several thousand filaments, must be able to be easily and completely wetted in an impregnation process with the relevant matrix resin. However, as carbon fibers are of low ductility and are brittle, they become easily frayed due to mechanical friction and often exhibit a poor wettability in regard to the employed matrix resins. To improve this, carbon fibers that are used as reinforcing materials for fiber-reinforced composites are usually pretreated with a sizing agent.

Carbon fibers that are treated with the inventive benzoxazine (co)polymer, for example in the form of an aqueous solution, emulsion or dispersion, are characterized by an improved handling in the manufacturing process for fiber-reinforced composites. Moreover, the treated carbon fibers exhibit an improved wettability with the relevant matrix resin. The wettability of the carbon fibers is particularly improved for benzoxazine-based resin systems. A further subject matter of the present invention is therefore the use of the inventive benzoxazine (co)polymers as a sizing agent, in particular as a sizing agent for carbon fibers. The inventive benzoxazine (co)polymers can likewise be used as a sizing agent for textile fibers or textile fabrics.

In the context of the present invention, the hard surfaces that are treated with the inventive benzoxazine (co)polymers, are particularly preferably selected from porcelain, glass, ceramic, plastic and/or metal.

The thus-treated surfaces are observed to possess an improved corrosion resistance. A further subject matter of the present invention is therefore the use of the inventive benzoxazine (co)polymers as a corrosion protection agent.

In the context of the present invention, the textile surfaces or hard surfaces that are treated with the inventive benzoxazine (co)polymer can be selected from textile fabrics or from the abovementioned hard surfaces.

Particularly preferred textile surfaces are textile fabrics made of wool, silk, hemp, cotton, linen, sisal, ramie, rayon, cellulose ester, polyvinyl derivatives, polyolefins, polyamides, viscose or polyester or their mixtures. In the context of the present invention, textile surfaces made of cotton or mixed cotton fabrics are quite particularly preferred.

The thus treated textiles or hard surfaces are characterized by a reduced redeposition of soils and by an improved soil removability. A further subject matter of the present invention is therefore is the use of the benzoxazine (co)polymers according to the invention for improving the soil removal from and/or reducing the redeposition of soils on textiles or hard surfaces.

After treatment of the hard surfaces with the inventive benzoxazine (co)polymer, due to the latter's particular chemical structure the surfaces exhibit a lower contamination from harmful microorganisms than do untreated surfaces. A further subject matter of the present invention is therefore the use of the inventive benzoxazine (co)polymers for coating surfaces, in particular for the antibacterial coating of surfaces.

### EXAMPLES

### 1. Preparation of Inventive Polymerizable Benzoxazine Compounds

The preparation of various polymerizable benzoxazine compounds of the Formula (B-Box-I) is described below:

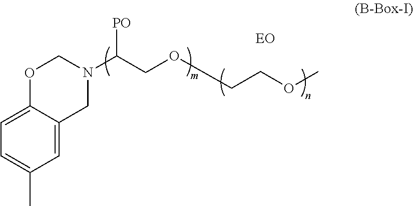

1.1 Preparation of a Polymerizable Benzoxazine Compound with the Use of Jeffamin M2070 (PO/EO 10/31); Designation (B-Box-I-1.1)

Starting materials:

| | |
|---|---|
| 9.38 g Paraformaldehyd (96% conc.) | 0.30 mol |
| in 50 ml Ethyl acetate | |
| 309.9 g Jeffamin M2070 (Huntsman) | 0.15 mol |
| in 200 ml Ethyl acetate | |
| 16.22 g p-Cresol | 0.15 mol |
| in 50 ml Ethyl acetate | |

The p-cresol, dissolved in ethyl acetate, was added drop wise over a period of 10 minutes to the solution of paraformaldehyde in ethyl acetate. Jeffamin M-2070 was then added over a period of 30 minutes, the temperature being maintained below 10° C. After stirring for 10 minutes, the reaction mixture was heated under reflux for 6 h. After cooling, the reaction mixture was filtered and the solvent together with any formed water were removed under vacuum. 318.90 g of the corresponding polymerizable benzoxazine compound were obtained.

1.2 Preparation of a Polymerizable Benzoxazine Compound with the Use of Jeffamin M 1000 (PO/EO 3/19); Designation (B-Box-I-1.2)

Starting materials:

| | |
|---|---|
| 18.7 g Paraformaldehyd (96% conc.) | 0.60 mol |
| in 50 ml Ethyl acetate | |
| 312.9 g Jeffamin M1000 (Huntsman) | 0.30 mol |
| in 250 ml Ethyl acetate | |
| 32.44 g p-cresol | 0.30 mol |
| in 60 ml Ethyl acetate | |

The p-cresol, dissolved in ethyl acetate, was added drop wise over a period of 10 minutes to the solution of paraformaldehyde in ethyl acetate. Jeffamin M-1000 was then added over a period of 30 minutes, the temperature being maintained below 10° C. After stirring for 10 minutes, the reaction mixture was heated under reflux for 6 h. After cooling, the reaction mixture was filtered and the solvent together with any formed water were removed under vacuum. 352.57 g of the corresponding polymerizable benzoxazine compound were obtained.

1.2 Preparation of Additional Polymerizable Benzoxazine Compounds with the Use of N-(3-aminopropyl)imidazole

The preparation of a polymerizable benzoxazine compound of the Formula (B-Box-II) is described below:

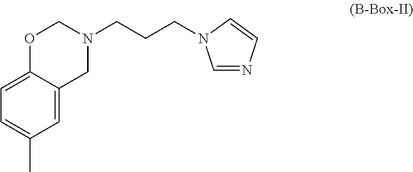 [SMILES: Cc1ccc2c(c1)CN(CCCn1ccnc1)CO2]

Starting materials:

| | |
|---|---|
| 78.20 g Paraformaldehyd (96% conc.) | 2.50 mol |
| in 100 ml Ethyl acetate | |
| 157.5 g N-(3-aminopropyl)imidazole (Lupragen API, BASF SE) | 1.25 mol |
| in 10 ml Ethyl acetate | |
| 135.17 g p-cresol | 1.25 mol |
| in 100 ml Ethyl acetate | |

The p-cresol, dissolved in ethyl acetate, was added drop wise over a period of 10 minutes to the solution of paraformaldehyde in ethyl acetate. Lupragen-API was then added over a period of 30 minutes, the temperature being maintained below 10° C. After stirring for 10 minutes, the reaction mixture was heated under reflux for 6 h. After cooling, the reaction mixture was filtered and the solvent together with any formed water were removed under vacuum. 322.74 g of the corresponding polymerizable benzoxazine compound were obtained.

1.3 Preparation of Additional Polymerizable Benzoxazine Compounds with the Use of Ethanolamine

The preparation of a polymerizable benzoxazine compound of the Formula (B-Box-III) is described below:

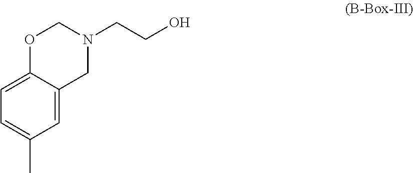

Starting materials:

| | |
|---|---|
| 106.35 g Paraformaldehyd (96% conc.) | 3.40 mol |
| in 100 ml Ethyl acetate | |
| 103.87 g ethanolamine | 1.70 mol |
| in 30 ml Ethyl acetate | |
| 183.84 g p-cresol | 1.70 mol |
| in 80 ml Ethyl acetate | |

The p-cresol, dissolved in ethyl acetate, was added drop wise over a period of 10 minutes to the solution of paraformaldehyde in ethyl acetate. Ethanolamine was then added over a period of 30 minutes, the temperature being maintained below 10° C. After stirring for 10 minutes, the reaction mixture was heated under reflux for 6 h. After cooling, the reaction mixture was filtered and the solvent together with any formed water were removed under vacuum. 328.6 g of the corresponding polymerizable benzoxazine compound were obtained.

### 2. Polymerization for the Preparation of a Benzoxazine (Co)Polymer

The above described polymerizable benzoxazine compounds were thermally cured as mixtures or individually in molds in an air circulating drying oven for a period of 2 h at 180° C. The samples were then removed from the molds and cooled down to room temperature. In this way benzoxazine (co)polymers were prepared in the compositions shown in Table 1.

TABLE 1
The contents of the polymerizable benzoxazine compounds in the
benzoxazine copolymers

| | Weight fraction of the respective polymerizable | | | |
|---|---|---|---|---|
| Benzoxazine | Benzoxazine compounds in % | | | |
| (co)polymer | B-Box-I-1.2 | B-Box-I-1.1 | B-Box-II | B-Box-III |
| 1 | 100 | | | |
| 2 | | 100 | | |
| 3 (Ref.) | | | 100 | |
| 4 (Ref.) | | | | 100 |
| 5 | | 30 | | 70 |
| 6 | | 50 | | 50 |
| 7 | 30 | | | 70 |
| 8 | 50 | | | 50 |
| 9 | | 30 | 70 | |
| 10 | | 50 | 50 | |
| 11 | 30 | | 70 | |
| 12 | 50 | | 50 | |
| 13 | | 30 | 35 | 35 |
| 14 | | 50 | 25 | 25 |
| 15 | 30 | | 35 | 35 |
| 16 | 50 | | 25 | 25 |

### 3. Solubility Behavior of the Benzoxazine (Co)Polymers

At least 0.1 g of a benzoxazine (co)polymer that was dried under vacuum was weighed out with at most 9.9 g of water (pH=7) in a 25 ml screw top vial. The mixture was then stirred (magnetic stirrer) at 70° C. for at least 5 minutes and then stirred at 22° C. for a further 45 minutes. Under these conditions, the benzoxazine (co)polymers of the present invention were taken up in an amount of at least 10 g/1000 g water without turbidity in water.

The solubility of the individual benzoxazine (co)polymers is shown in Table 2.

TABLE 2
Solubility of some benzoxazine (co)polymers in water

| Benzoxazine (co)polymer | Solubility in water |
|---|---|
| 1 | + |
| 2 | + |
| 3 (ref) | − |
| 4 (ref) | − |
| 8 | + |
| 12 | + |
| 16 | + |

+ solubility > 10 g benzoxazine (co)polymer/1000 g water
− solubility < 10 g benzoxazine (co)polymer/1000 g water

As is illustrated in Table 2, under the cited conditions, the non-inventive benzoxazine (co)polymers 3 and 4 exhibit a poorer solubility behavior in water than the inventive benzoxazine (co)polymers 1, 2, 8, 12 and 16.

1-13. (canceled)

14. A benzoxazine compound selected from one or both of: 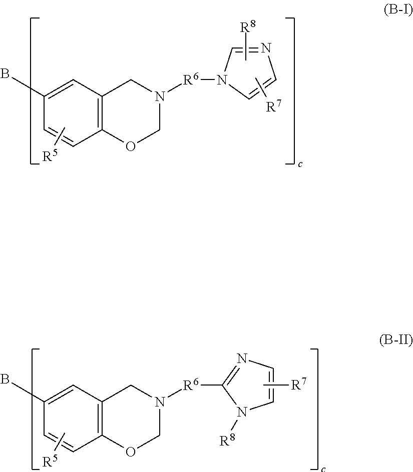
wherein c is 1 to 4; B is selected from hydrogen (for c=1), alkyl (for c=1), alkylene (for c=2 to 4), carbonyl (for c=2), oxygen (for c=2), sulfur (for c=2), sulfoxide (for c=2), sulfone (for c=2) and a direct, covalent bond (for c=2), A is a hydroxyl group or a nitrogen-containing heterocycle, R⁵ is selected from hydrogen, halogen, alkyl and alkenyl, or R⁵ is a divalent group that makes a corresponding naphthoxazine structure from the benzoxazine structure and, R⁶ stands for a covalent bond or a divalent linking group that contains 1 to 100 carbon atoms; and R⁷ and R⁸ are each independently of one another are selected from hydrogen, halogen, linear or branched, optionally substituted alkyl groups, alkenyl groups and aryl groups.

15. The benzoxazine compound of claim 14 selected from the group consisting of:

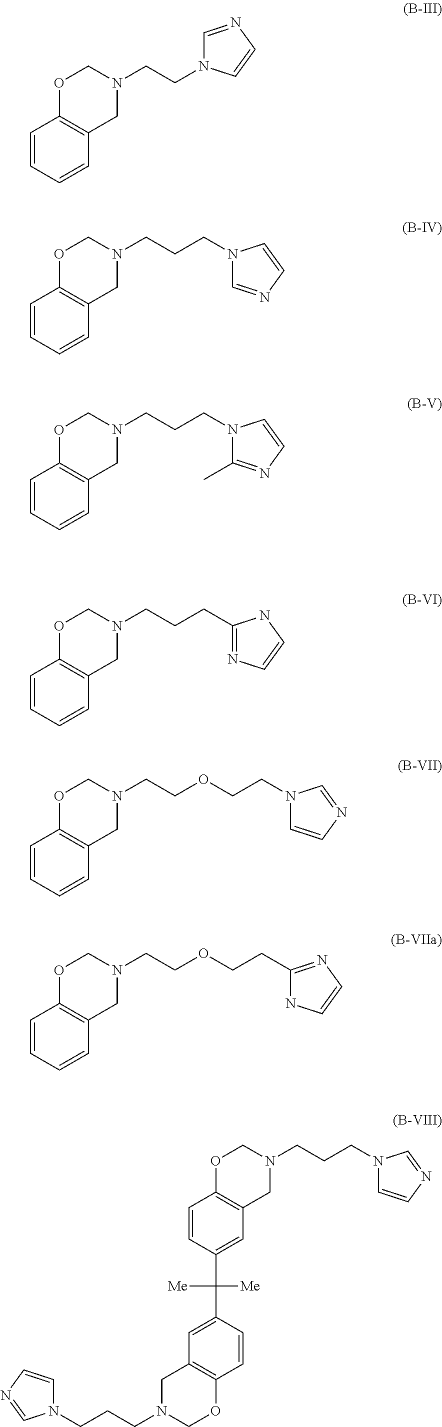

16. A benzoxazine (co)polymer, wherein the benzoxazine (co)polymer in polymerized form contains at least one polymerizable benzoxazine compound of claim 14.

17. The benzoxazine (co)polymer according to claim 16, wherein the benzoxazine (co)polymer has a weight average molecular weight of 500 to 100 000 g/mol.

18. The (co)polymer according to claim 16 and water.

19. A fiber-reinforced composite comprising:
A matrix resin; and
Carbon fiber treated with a sizing agent comprising a benzoxazine compound of claim 14.
